(19) United States
(12) Patent Application Publication
(10) Pub. No.: US 2025/0367105 A1
(43) Pub. Date: Dec. 4, 2025

(54) Makeup and/or Care Composition Comprising a Volatile Solvent, a Particular Polyester, an Alkylcellulose and a Dyestuff, and Process Using Same

(71) Applicant: L'OREAL, Paris (FR)

(72) Inventors: PASCAL ARNAUD, Chevilly Larue (FR); NATHALIE GUILLIER, Chevilly Larue (FR)

(73) Assignee: L'OREAL, Paris (FR)

(21) Appl. No.: 19/121,860

(22) PCT Filed: Oct. 10, 2023
(86) PCT No.: PCT/EP2023/078039

§ 371 Date: Apr. 17, 2025

(30) Foreign Application Priority Data
Oct. 19, 2022 (FR) FR2210778

(51) Int. Cl. A61K 8/85; A61K 8/31; A61K 8/34; A61K 8/36; A61K 8/73; A61Q 1/02; A61Q 1/04

(52) U.S. Cl. CPC A61K 8/85; A61K 8/31; A61K 8/34; A61K 8/361; A61K 8/731; A61Q 1/02; A61Q 1/04; A61K 2800/43

(57) ABSTRACT

The present invention relates to a composition for making up and/or caring for the skin and/or the lips, comprising: A) at least one volatile solvent; B) at least one polyester which is the product of reaction of components (i), (ii) and (iii) below: (i) at least one polyglycerol-3; (ii) at least one acid dimer; and (iii) at least one fatty monoacid containing from 8 to 30 carbon atoms, components (i), (ii) and (iii) reacted being in a mole ratio of 1 mol of polyglycerol-3, 0.5 to 1 mol of acid dimer and from 0.1 to less than 2.0 mol of fatty monoacid; and C) at least one alkylcellulose, the alkyl group of which is C2-C3, preferably ethylcellulose; D) at least one dyestuff. The invention also relates to a process for making up and/or caring for the skin and/or the lips, which consists in applying said composition.

The subject of the present invention is a makeup and/or care composition for the skin and/or lips, comprising a volatile solvent, a particular polyester, an alkylcellulose and a dyestuff, and also a makeup and/or care process for human keratin materials using same.

Many cosmetic makeup compositions containing dyestuffs, such as foundations, correctors, lipsticks or lip glosses, have been developed to improve the staying power and transfer resistance properties. Specifically, poor staying power may be reflected in particular by poor persistence of the colour (changing or fading) and/or of the gloss of the deposit. This consequently obliges the user to freshen the makeup more regularly than desired, which may be considered as time consuming.

Improving the staying power of compositions is obtained by means of compositions which form a film after application. Such compositions generally contain volatile solvents which evaporate on contact with the skin or the lips, leaving behind a layer comprising waxes and/or film-forming polymers, pigments and fillers. Film-forming polymers are synthetic polymers, usually silicone or acrylic polymers. Thus, mention may be made of the use of silicone resins, for instance trimethyl siloxysilicate (INCI name) or polypropylsilsesquioxane (INCI name) resins, or resins which comprise silicone polymers such as silicone acrylate dendrimer copolymers (acrylates/polytrimethyl siloxymethacrylate copolymer—INCI name). Acrylic polymers such as acrylic acid/isobutyl acrylate/isobornyl acrylate copolymers are also used. However, these compositions are often considered less comfortable, or even uncomfortable, from a sensory point of view for consumers.

Moreover, in recent years, consumers have become increasingly demanding regarding the composition of their cosmetic products and are in particular seeking to use products with a larger content of natural ingredients or ingredients of natural origin, ingredients whose environmental impact is minimized and/or ingredients that are compatible with a wide range of packaging.

The difficulty remains, however, in reconciling these latest trends with the fact that consumers do not, however, want to give up the very high performance to which they have become accustomed regarding the products they already use.

It has already been proposed, in makeup compositions of the prior art, to use liquid or pasty polyesters to obtain staying power properties.

Mention may notably be made of JP2002-128623, JP2002-128628, JP2002-128629 and EP 1 604 634, which describe polyesters of dilinoleic diacids and dilinoleyl diol dimers having the INCI name Dimer Dilinoleyl Dimer Dilinoleate, such as those sold by the company Nippon Fine Chemical under the trade names Lusplan DD-DA5® and DD-DA7®.

To obtain staying power properties in makeup compositions, it has also been proposed, in FR2931673, to use polyesters obtained by condensation of dimer and/or trimer of unsaturated fatty acids and of diol; in particular, the polyester obtained by condensation of dimer and/or trimer of unsaturated fatty acid and of diol is a polyester of dilinoleic acid and of 1,4-butanediol, such as the polymer sold by Biosynthis under the name Viscoplast 14436H® (INCI name: Dilinoleic Acid/Butanediol Copolymer).

It is also known practice, notably in FR 2 931 069, JP2005-325079 and JP2006-28129, to use polyesters of hydroxylated fatty acid triglyceride and of a saturated fatty diacid to provide staying power to makeup compositions. As examples of polyesters, mention may be made in particular of those having the INCI name Hydrogenated Castor Oil/Sebacic Acid Copolymer, such as the product sold under the name Crodabond CSA® by the company Croda, and also the hydrogenated castor oil dimer dilinoleate having the INCI name: Hydrogenated Castor Oil Dimer Dilinoleate, such as the product sold under the names Risocast-DA-L® and Risocast DA-H® by the company Kokyu Alcohol Kogyo.

However, it was found that liquid makeup compositions comprising the polyesters mentioned previously were not fully satisfactory and could lead to unsatisfactory film adhesion and makeup removal results. These polyesters can result, on application, in dewetting or in a film which runs between the two lips after the lips have been pressed together (blotting). Finally, it may occasionally be observed that the films break down into pieces on makeup removal.

The need remains to find novel compositions for making up and/or caring for the skin and/or the lips, based on a suitable polyester, leading to the production of a glossy deposit, with good staying power of the deposit and good transfer resistance properties, without the drawbacks mentioned previously.

It has unexpectedly been discovered that these objects may be achieved with a composition for making up and/or caring for the skin and/or lips, preferably the lips, notably comprising, in a physiologically acceptable medium:
A) at least one volatile solvent;
B) at least one polyester which is the product of reaction of components (i), (ii) and (iii) below:
(i) at least one polyglycerol-3;
(ii) at least one acid dimer; and
(iii) at least one fatty monoacid containing from 8 to 30 carbon atoms, components (i), (ii) and
(iii) reacted being in a mole ratio of 1 mol of polyglycerol, 0.5 to 1 mol of acid dimer and 0.1 to 2.0 mol of fatty monoacid;
C) at least one alkylcellulose, the alkyl group of which is C2-C3, preferably ethylcellulose;
D) at least one dyestuff.

A subject of the invention is also a process for making up and/or caring for the skin and/or the lips, preferably the lips, which consists in applying the composition described previously.

The composition according to the invention has the advantage of being stable over time and of being easy to apply, without dewetting on application or on blotting. Moreover, the deposit obtained is precise, homogeneous, not runny and sparingly or not at all tacky. The deposit does not migrate into the wrinkles and fine lines, in particular around the lips.

The resulting deposit is glossy, with improved staying power. It is also comfortable, without leaving a feeling of dryness or tautness.

### Definitions

Since the composition according to the invention is a cosmetic composition, this means in particular that it comprises a physiologically acceptable medium, suitable for the nature of the support onto which the composition is to be applied, and also for the form in which the composition is to be packaged.

The term “physiologically acceptable” means compatible with the skin and/or the lips, which has a pleasant colour, odour and feel, and which does not cause any unacceptable discomfort (stinging or tautness) liable to discourage the consumer from using this composition.

The term “liquid composition” means any composition which has at least one of the following characteristics:
i) it flows under its own weight at room temperature (20° C.) and at atmospheric pressure (1.013×10⁵ Pa);
ii) it is not solid at room temperature and at atmospheric pressure and a viscosity or consistency characterized by its hardness may be measured for same;
iii) it does not have any particular shape such as that which can be obtained by hot casting in a mould or container of a given shape.

Such compositions may thus be found notably in fluid, creamy, pasty or gel form.

The term “polyester” means any polymer obtained by a condensation reaction of polycarboxylic acids with alcohols or polyols. Its macromolecular backbone contains the repetition of its ester function. The ester function denotes a characteristic group formed of an atom bonded simultaneously to an oxygen atom via a double bond and to an alkoxy group.

When the bonded atom is a carbon atom, it is called a carboxylic ester, the general formula of which is R—COO—R′.

The term “polyglycerol-3” means triglycerol alone or a mixture of polyglycerols comprising at least triglycerol, and preferably triglycerol is predominant in said mixture.

### Protocol for Measuring the Viscosity:

The viscosity measurement is generally performed at 25° C., using a Rheomat RM180 viscometer equipped with a No. 2, 3 or 4 spindle, the measurement being performed after 10 minutes of rotation of the spindle in the composition (after which time stabilization of the viscosity and of the spin speed of the spindle are observed), at a shear rate of 200 rpm.

According to one embodiment, the composition according to the invention may have, at 25° C., a viscosity of between 0.1 and 25 Pa·s, preferably between 0.2 and 20 Pa·s.

Preferably, the viscosity at 25° C. of a composition according to the invention may be between 0.2 and 10 Pa·s. In particular, the viscosity at 25° C. of a composition according to the invention may be between 0.1 Pa·s (spindle 2) and 25 Pa·s (spindle 4), preferably between 0.2 Pa·s (spindle 2) and 20 Pa·s (spindle 4) and better still between 0.2 Pa·s (spindle 2) and 10 Pa·s (spindle 4).

### Volatile Solvents

The composition in accordance with the present invention comprises at least one volatile solvent.

In the context of the invention, the term “volatile solvent” means a compound that is liquid at room temperature (20° C.) and at atmospheric pressure (1.013×10⁵ Pa), having a vapour pressure at 20° C. of greater than 2.66 Pa, preferably between 2.66 and 40 000 Pa, in particular up to 27 000 Pa.

Among the volatile solvents, mention may be made of:
monoalcohols including from 2 to 6 carbon atoms;
volatile oils chosen from apolar hydrocarbon-based oils and silicone oils, or mixtures thereof;
mixtures thereof.

According to a particular form of the invention, the volatile solvent is chosen from:
monoalcohols including from 2 to 6 carbon atoms;
apolar hydrocarbon-based volatile oils, or mixtures thereof;
mixtures thereof.

### C2-C6 Monoalcohols

The monoalcohol(s) in accordance with the invention preferably comprise from 2 to 6 carbon atoms and in particular from 2 to 4 carbon atoms, and mixtures thereof.

The monoalcohol(s) may be represented, for example, by the formula RaOH, in which Ra represents a linear or branched alkyl group comprising from 2 to 6 carbon atoms.

Monoalcohols that may be mentioned include ethanol, isopropanol, propanol or butanol, and more particularly ethanol.

According to an advantageous embodiment, the amount of monoalcohol(s) ranges from 2% to 60% by weight, preferably from 4% to 50% by weight and even more preferentially from 6% to 40% by weight relative to the total weight of said composition.

### Volatile Oils

The term “oil” means any lipophilic compound that is in liquid form at room temperature and at atmospheric pressure.

The volatile oil(s) are chosen from apolar hydrocarbon-based oils and silicone oils, or mixtures thereof.

For the purposes of the invention, the term “volatile oil” refers to any oil that is capable of evaporating on contact with the skin in less than one hour, at room temperature and atmospheric pressure. The volatile oil is a volatile cosmetic compound, which is liquid at room temperature, notably having a non-zero vapour pressure, at room temperature and atmospheric pressure, notably having a vapour pressure ranging from 2.66 Pa to 40 000 Pa, in particular ranging from 2.66 Pa to 13 000 Pa and more particularly ranging from 2.66 Pa to 1300 Pa.

The term “apolar hydrocarbon-based oil” means an oil chosen from hydrocarbons, that is to say from compounds comprising only carbon and hydrogen atoms.

The term “silicone oil” denotes an oil comprising at least one Si—O group, and more particularly an organopolysiloxane.

The apolar hydrocarbon-based volatile oils that may be used in the context of the invention are more particularly chosen from linear or branched, preferably saturated, oils containing from 6 to 16 carbon atoms, and mixtures thereof.

The volatile hydrocarbon-based oils that may be used in the compositions according to the invention may thus be chosen from volatile linear alkanes comprising from 6 to 14 carbon atoms.

As examples of linear alkanes, in particular C6-C14 alkanes, mention may be made of n-hexane (C6), n-heptane (C7), n-octane (C8), n-nonane (C9), n-decane (C10), n-undecane (C11), n-dodecane (C12) and n-tridecane (C13), and mixtures thereof. Mention may notably be made of n-dodecane (C12) and n-tetradecane (C14) sold by Sasol under the references, respectively, Parafol 12-97® and Parafol 14-97®, and also mixtures thereof. According to another embodiment, use may be made of a mixture of n-dodecane and n-tetradecane, and in particular the dodecane/tetradecane mixture sold by the company Biosynthis under the reference Vegelight 1214®. According to yet another embodiment, use may also be made of a mixture of volatile linear C9-C12 alkanes of INCI name: C9-12 Alkane, such as the product sold by the company Biosynthis under the reference Vegelight Silk®. According to yet another embodiment, use may be made of a mixture of n-undecane (C11) and of n-tridecane (C13) as obtained in Examples 1 and 2 of patent application WO 2008/155 059 from the company Cognis and the product sold under the trade name Cetiol Ultimate® by the company BASF.

Mention may also be made of the alkanes described in the Cognis patent applications WO 2007/068 371 or WO 2008/155 059 (mixtures of different alkanes differing by at least one carbon). These alkanes are obtained from fatty alcohols, which are themselves obtained from coconut kernel oil or palm oil.

The volatile hydrocarbon-based oils that may be used in the compositions according to the invention may be chosen from branched C8-C16 alkanes. Mention may notably be made of C8-C16 isoalkanes of petroleum origin (also known as isoparaffins), such as isododecane (also known as 2,2,4,4,6-pentamethylheptane), isodecane, isohexadecane and, for example, the oils sold under the Isopar® or Permethyl® trade names.

According to a particularly preferred embodiment, the volatile hydrocarbon-based oil is chosen from branched C8-C16 alkanes, and more particularly isododecane, the mixture of volatile linear C9-C12 alkanes and the mixture of n-undecane (C11) and of n-tridecane (C13) and mixtures thereof.

As examples of volatile silicone oils that can be used in the invention, mention may be made of volatile silicone oils, such as linear or cyclic volatile silicone oils notably containing from 2 to 7 silicon atoms, these silicones optionally including alkyl or alkoxy groups containing from 1 to 10 carbon atoms. As volatile silicone oils that may be used in the invention, mention may notably be made of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

Preferably, the volatile oil(s) are chosen from apolar volatile hydrocarbon-based oils, notably from branched C8-C16 alkanes, and more particularly isododecane, mixtures of volatile linear C9-C12 alkanes and the mixture of n-undecane (C11) and of n-tridecane (C13), and mixtures thereof.

The volatile oil(s) are preferably present in the composition of the invention in contents of less than 60% by weight and preferably less than 50% by weight relative to the total weight of said composition.

According to a preferential form of the invention, the weight ratio of the amount of volatile oil(s) to the amount of monoalcohol(s) is less than 3.5 and even more preferentially less than 1.5.

### Water

The composition according to the invention may optionally comprise water.

A water that is suitable for use in the invention may be a demineralized water, a floral water such as cornflower water and/or a mineral water such as Vittel water, Lucas water or La Roche Posay water and/or a spring water.

According to a particular embodiment of the invention, if the composition according to the invention comprises water, its content is less than 5% by weight, more particularly less than 2% by weight and preferably less than 0.5% by weight, relative to the total weight of the composition.

### Polyester of Polyglycerol/Acid Dimer/Monoacid

The composition in accordance with the invention contains at least one polyester which is the product of reaction of components (i), (ii) and (iii) below:
(i) at least one polyglycerol-3;
(ii) at least one acid dimer; and
(iii) at least one fatty monoacid containing from 8 to 30 carbon atoms, components (i), (ii) and (iii) reacted being in a mole ratio of 1 mol of polyglycerol-3, 0.5 to 1 mol of acid dimer and 0.1 to less than 2.0 mol of fatty acid.

The polyesters of the invention and the synthesis thereof are described in patent applications US 2021/0259945, US 2021/0259946 and US 2021/0259930.

According to a preferred embodiment, the amount of polyester active material ranges from 1% to 50% by weight, more preferentially from 3% to 20% by weight relative to the total weight of the composition.

According to a preferred embodiment, the polyester is a product of substantially or totally non-sequential reaction.

The term “product of substantially non-sequential reaction” means the product obtained by a substantially non-sequential reaction of the reactive components (i)-(iii).

The term “totally non-sequential reaction of the reactive components (i)-(iii)” means that the total content of each of the reagents (i)-(iii) to be made to react is added to the reaction vessel before starting the reaction.

In one embodiment of the present invention, the total content of each of the reagents (i)-(iii) to be made to react is added to the reaction vessel before starting the reaction, that is to say that the reaction is totally non-sequential, and the polymer is a product of totally non-sequential reaction of the components (i)-(iii). In other embodiments, 70-100%, or 75-100%, or 80-100%, or 85-100%, or 90-100%, or 95-100%, or 97-100% of each of the reagents (i)-(iii) are added to the reaction vessel before starting the reaction.

In one embodiment, the polyester is prepared by a one-step process which involves the introduction of all the reagents into a reaction vessel and the subsequent induction of an entirely random addition of the acid dimer and of isostearic acid to the polyglycerol-3.

Triglycerol has the formula H—[-OGly]3-OH in which Gly designates a glycerol residue after removal of two hydroxyl groups.

### Polyglycerol-3

A polyglycerol-3 according to the invention in the form of a mixture of polyglycerols containing at least triglycerol also comprises polyglycerols which can be any product of oligocondensation of glycerol. They preferably correspond to formula (I) below: H[—O-Gly-]n-OH, in which each Gly is independently the residue of a glycerol molecule after removal of two hydroxyl groups; and n is a mean from 2 to 10.

Generally, the majority of the Gly groups are of the formula: —CH₂—CHOH—CH₂—, although residues comprising etherification at secondary or even tertiary hydroxyl groups are regarded as being within the scope of “Gly” and, consequently, may also be present.

Examples of polyglycerol-3 comprise diglycerol, triglycerol, tetraglycerol, pentaglycerol, hexaglycerol, heptaglycerol, octaglycerol, nonaglycerol, decaglycerol and mixtures thereof. In particular, preferential polyglycerols are those of formula (I) in which n in particular has a value from 2 to 7, more particularly from 2 to 5 and notably 2, 3 or 4, or mixtures of polyglycerols in these ranges.

Particularly appropriate examples of polyglycerol-3 comprise a mixture of polyglycerols having the following distribution, in which all the weight percentages are based on a total content of the polyglycerol-3 in the form of a mixture:
glycerol: 0% to 30% by weight, preferably 0% to 20% by weight, most preferably 0% to 15% by weight;
diglycerol: 10% to 40% by weight, preferably 15% to 35% by weight, most preferably 20% to 32% by weight;
triglycerol: 10% to 65% by weight, preferably 15% to 60% by weight, most preferably 18% to 55% by weight;
tetraglycerol: 2% to 25% by weight, preferably 5% to 20% by weight, most preferably 8% to 20% by weight;
pentaglycerol: 0% to 15% by weight, preferably 0% to 10% by weight, most preferably 0% to 5% by weight;
hexaglycerol: 0% to 15% by weight, preferably 0% to 10% by weight, most preferably 0% to 5% by weight;
heptaglycerol: 0% to 10% by weight, preferably 0% to 5% by weight, most preferably 0% to 3% by weight;
octaglycerol: 0% to 10% by weight, preferably 0% to 5% by weight, most preferably 0% to 3% by weight;
nonaglycerol: 0% to 5% by weight, preferably 0% to 3% by weight, most preferably 0% to 2% by weight;
decaglycerol: 0% to 5% by weight, preferably 0% to 3% by weight, most preferably 0% to 2% by weight.

In one embodiment, the polyglycerol-3 comprises the following distribution of polyglycerols:
Glycerol: 0% to 30% by weight;
Diglycerol: 15% to 40% by weight;
Triglycerol: 10% to 55% by weight;
Tetraglycerol: 2% to 25% by weight;
Pentaglycerol and higher components: 0% to 15% by weight,
all the weight percentages being based on a total content of polyglycerol-3 in the form of a mixture.

In one embodiment, the polyglycerol-3 is composed of at least 40% by weight, or of at least 45% by weight, or of at least 50% by weight, relative to the total weight of the polyglycerol-3, of a combination of diglycerol and of triglycerol.

In one embodiment, the polyglycerol-3 is composed of at least 20% by weight, or of at least 25% by weight, of diglycerol; at least 15% by weight, or at least 18% by weight, of triglycerol; at least 10% by weight, or at least 12% by weight, of tetraglycerol; in which all the weight percentages are based on a total content of the polyglycerol-3 in the form of a mixture.

A particularly preferred polyglycerol-3 comprises at least 25% by weight of diglycerol, at least 45% by weight of triglycerol and at least 10% by weight of tetraglycerol, relative to the total weight of the polyglycerol-3 in the form of a mixture.

The analysis of such a polyglycerol-3 composition may be performed so as to determine its median or “mean” polyglycerol number. The above examples of polyglycerols with narrow and broad distributions can also be denoted as polyglycerol-3 because it is a matter of the integer closest to the mean and/or median.

### Acid Dimer

The acid dimer may be any dicarboxylic acid containing at least 4 carbon atoms. They may be linear or branched, for instance the dimers prepared from malonic acid, succinic acid, fumaric acid, dimethylglutaric acid or trimethyladipic acid, and from anhydrides thereof.

Fatty acid dimers are particularly useful. As is known, these are mixtures of acyclic and cyclic dicarboxylic acids which are obtained by a catalysed dimerization reaction of unsaturated fatty acids containing from 12 to 22 carbon atoms.

For the preparation and use of acid dimers and their physical and chemical properties, reference will be made to the publication “The Dimer Acids: The Chemical and Physical Properties, Reactions and Applications”, Ed. E. C. Leonard; Humko Sheffield Chemical, 1975, Memphis, Tenn.

The dicarboxylic acids may also contain, to a lesser extent, tri- and polyfunctional carboxylic acids. The functionality of the mixture must not exceed a mean molar value of 2.4.

The preferred acid dimers are typically derived from triglycerides rich in C18 ester groups, which can be hydrolysed to produce unsaturated C18 fatty monoacids. The starting materials may be derived from tallow oil and rapeseed oil but other natural sources, such as flax seeds, soybean, pumpkin and walnut, may be used. The target monoacids used in the reaction are rich in forms of oleic and linoleic acids which are described in the list of fatty acids contained below. Dimerization results mainly in the dimerization of unsaturated fatty acids, but trimers are also formed. After reaction, the product may be stored in the form of a mixture of reaction products or it may be further distilled or otherwise separated into molecular weight fractions. In one embodiment, the dimerization reaction produces a predominance (at least 60% by weight, more preferably at least 75% by weight) of acid dimer (C36 diacid) but also produces C54 acid trimers (less than 30% by weight, more preferably less than 25% by weight).

In one case, a standard acid dimer commercially available from Croda, Pripol 1025®, which contains 72% by weight of acid dimer and 19% by weight of acid trimer, is used.

In another case, a standard hydrogenated acid dimer from Oleon, Radiacid 0960®, which contains 87% by weight of acid dimer and 10% by weight of acid trimer, is used. In both cases, the polymer as described is characterized by a higher molecular weight, a more hydrophobic nature and a higher viscosity than those which can be provided by pure diacids of lower molecular weight. The presence of acid trimer further improves the molecular weight and the performance qualities of these polymers.

In one embodiment, the copolymer of the present invention is prepared from at least one hydrogenated acid dimer.

In another embodiment, the polymer is prepared from a hydrogenated acid dimer comprising hydrogenated dimerized C18 fatty acids, which hydrogenated acid dimer is obtained by dimerization of unsaturated C18 fatty acids and subsequent hydrogenation.

In one embodiment, the hydrogenated acid dimer contains a content of acid trimer ranging from about 5% to 25% by weight, based on the total weight of hydrogenated acid dimer.

In another embodiment, the hydrogenated acid dimer contains a predominance (at least 60% by weight, more preferentially at least 75% by weight, but not more than 95% by weight, or better still not more than 90% by weight, or even better still not more than 85% by weight) of hydrogenated acid dimer (C36 diacid) and also contains hydrogenated C54 acid trimers (less than 30% by weight, more preferably less than 25% by weight, but more than 5% by weight, more preferably more than 10% by weight).

### C8-C30 Fatty Monoacid

The C8-C30 fatty monoacids may include natural or refined fatty acids, such as hydrolysed rapeseed oil, hydrolysed sunflower oils, and the like, but these contain both lower and higher MW chains. Useful fatty monoacids may be linear, branched, saturated, unsaturated and aromatic materials with an acidity provided by carboxylic acid fractions.

Acids that are suitable for use in the invention comprise caprylic acid (C8), pelargonic acid (C9), capric acid (C10), undecylic acid (C11), lauric acid (C12), tridecylic acid (C13), myristic acid (C14), pentadecylic acid (C15), palmitic acid (C16), margaric acid (C17), stearic acid (C18), isostearic acid (C18), nonadecylic acid (C19), arachidic acid (C20), behenic acid (C22) and lignoceric acid (C24).

The comparison of stearic acid and isostearic acid shows that the branching leads to an elevated melting point and results in a low viscosity at room temperature for isostearic acid, compared to a solid material for stearic acid. This lower viscosity can be useful in the handling of starting materials and also to make it possible for the esters manufactured with this acid to retain their liquid properties. Branched-chain fatty acids often contain a single methyl branch along the linear carbon chain and are produced in nature by microbial action. Isostearic acid is available as a reaction by-product in the creation of the acid dimer described above.

Another way to obtain a liquid product consists in using unsaturated, linear and branched, fatty monoacids. These unsaturated acids may include palmitoleic acid (C16:1), vaccenic acid (C18:1), oleic acid (C18:1), elaidic acid (C18:1), linoleic acid (C18:2), linolelaidic acid (C18:2), α-linolenic acid (C18:3), γ-linolenic acid (C18:3), stearidonic acid (C18:4), paullinic acid (C20:1), gondoic acid (C20:1), dihomo-γ-linolenic acid (C20:3), mead acid (C20:3), arachidonic acid (C20:4), eicosapentaenoic acid (C20:5), erucic acid (C22:1), docosatetraenoic acid (C22:4), cervonic acid (C22:6) and nervonic acid (C24:1). As is well known to a person skilled in the art, the designation means that the length of the carbon chain is X carbon atoms and that there are Y double bonds in the chain.

In one embodiment, isostearic acid will be preferred.

In a particularly preferred embodiment, the polyester of the invention is a product of substantially or totally non-sequential reaction of the following components:
(i) at least one polyglycerol-3 comprising at least 25% by weight of diglycerol, at least 45% by weight of triglycerol and at least 10% by weight of tetraglycerol, in each case relative to the total weight of polyglycerol-3 in the form of a mixture;
(ii) at least one hydrogenated acid dimer containing at least 60% by weight of hydrogenated C36 diacid and from 5% to 25% by weight of hydrogenated C54 triacid, in each case relative to the total weight of hydrogenated acid; and
iii) isostearic acid.

In one embodiment, the polyester is prepared by a one-step process which involves the introduction of all the reagents into a reaction vessel and the subsequent induction of an entirely random addition of the acid dimer and of isostearic acid to the polyglycerol-3.

In one embodiment, it is preferable to have a total degree of esterification of the available polyglycerol hydroxyl fragments (total esterification) of from 24% to 74% and a degree of esterification of the available polyglycerol hydroxyl fragments by an acid dimer alone (esterification with an acid dimer) of from 20% to 40%. Even more importantly, the degree of esterification by end-cap units (esterification with a monoacid) is also defined in this description and it is important to maintain the esterification with a monoacid from 4% to 40%.

It is preferable to have a total esterification of 28% to 57% with an esterification with an acid dimer of 20% to 30% and an esterification with a monoacid of between 8% and 27%.

It is even more preferable to have a total esterification of 33% to 48% with an esterification with an acid dimer of 20% to 28% and an esterification with a monoacid of between 13% and 20%.

It is even more preferable to have a total esterification of 24% to 74% with an esterification with a hydrogenated acid dimer of 20% to 40% and an esterification with a monoacid of between 4% and 40%.

It is even more preferable to have a total esterification of 28% to 57% with an esterification with a hydrogenated acid dimer of 20% to 30% and an esterification with a monoacid of between 8% and 27%.

It is also even more preferable to have a total esterification of about 40% with an esterification with a hydrogenated acid dimer of about 20% and an esterification with a monoacid of about 20%.

It is also even more preferable to have also most preferably a total esterification of about 40% with an esterification with a hydrogenated acid dimer of about 27% and an esterification with a monoacid of about 13%.

In one embodiment, the reacted components are in a mole ratio of 1 mol of polyglycerol-3, 0.5 to 1 mol of acid dimer and 0.2 to 1.7 mol of fatty acid.

In another embodiment, the reacted components are in a mole ratio of 1 mol of polyglycerol-3, 0.5 to 0.75 mol of acid dimer and 0.4 to 1.35 mol of isostearic acid.

In another embodiment, the reacted components are in a mole ratio of 1 mol of polyglycerol-3, 0.5 to 0.7 mol of acid dimer and 0.65 to 1 mol of isostearic acid.

In another embodiment, the reacted components are in a mole ratio of 1 mol of polyglycerol-3, 0.5 to 1 mol of hydrogenated acid dimer and 0.2 to 1.7 mol of isostearic acid.

In another embodiment, the reacted components are in a mole ratio of 1 mol of polyglycerol-3, 0.5 to 0.75 mol of hydrogenated acid dimer and 0.4 to 1.35 mol of isostearic acid.

In another embodiment, the reacted components are in a mole ratio of 1 mol of polyglycerol-3, 0.5 to 0.7 mol of hydrogenated acid dimer and 0.65 to 1 mol of isostearic acid.

In another embodiment, the reacted components are in a mole ratio of 1 mol of polyglycerol-3, 0.5 to 1 mol of hydrogenated acid dimer and 0.2 to 1.7 mol of isostearic acid.

In another embodiment, the reacted components are in a mole ratio of 1 mol of polyglycerol-3, 0.5 to 0.75 mol of hydrogenated acid dimer and 0.4 to 1.35 mol of isostearic acid.

In another embodiment, the reacted components are in a mole ratio of 1 mol of polyglycerol-3, 0.5 to 0.7 mol of hydrogenated acid dimer and 0.65 to 1 mol of isostearic acid.

In another embodiment, the reacted components are in a mole ratio of 1 mol of polyglycerol-3, 0.67 mol of hydrogenated C36 acid dimer and 0.67 mol of isostearic acid.

In a particularly preferred embodiment, the reacted components are in a mole ratio of 1 mol of polyglycerol-3, 0.5 mol of hydrogenated C36 acid dimer and 1 mol of isostearic acid.

By adjusting the mole ratio of the termination of the fatty acids and by balancing the amount of polyglycerol-3 and of acid dimer, it is also possible to control the degree of acid dimer-polyglycerol extension and termination so that crosslinking, for example, via the acid trimer, results in much higher viscosities.

The target viscosity of the pure polymer must be greater than 50 000 mPa·s and less than 5 000 000 mPa·s at 25° C.

In one preferred embodiment, the target viscosity is >75 000 mPa·s and <2 500 000 mPa·s at 25° C.

In another preferred embodiment, the target viscosity is >100 000 mPa·s and <2 000 000 mPa·s at 25° C.

In a most preferred embodiment, the target viscosity is >1 000 000 mPa·s and <2 000 000 mPa·s at 25° C.

The viscosity is measured using an MCR3O2® rheometer from Anton Paar Inc. Rough or smooth twin flat plates 50 mm in diameter were used, covered with a polymer sample, adjusted to a gap of 0.5 to 1 mm, and temperature and shear rate scans were performed. The polyesters of the invention have Newtonian behaviour and thus have a constant viscosity over a wide range of shear rates. In addition, the polymers of the present description demonstrated a reduced viscosity with temperature. Thus, the viscosity measurements are reported at a precisely controlled temperature and generally in the form of a shear rate of 1. The values are reported in mPa·s.

The polyesters of the invention are characterized by weight-average molecular masses >2500 Da and <1 000 000 Da, measured by GPC using linear polystyrene standards.

The GPC column used for these tests consisted of: Phenolgel, 300×4.6 mm; a continuous tetrahydrofuran (THF) phase was used and injected at 0.35 ml/min, column oven maintained at 40° C.; a 50 μl injection and a Wyatt Ri refractive index detector. The calibration standards used were strictly linear polystyrene intended to be monodispersed. The narrow range polystyrene GPC calibration standards were prepared as a mobile phase and had maximum molecular weights of 1 290 000 Da, 560 000 Da, 65 500 Da, 28 500 Da, 10 100 Da, 1680 Da, 580 Da and 208 Da. Using standard methodologies, the weight- and number-average molecular mass is automatically calculated with standard GPC software.

In a preferred embodiment, the polyesters described have a weight-average molecular weight >4000 Da and <250 000 Da, measured by GPC using linear polystyrene standards.

In a most preferred embodiment, the polymers described have a weight-average molecular weight >5000 Da and <150 000 Da, measured by GPC using linear polystyrene standards.

In yet another embodiment, the polyester of the invention has a combination of weight-average molecular mass >5000 Da and <150 000 Da, measured by GPC using linear polystyrene standards, and of viscosity at 25° C.>100 000 mPa·s and <2 000 000 mPa·s.

In a preferred embodiment, the polyester of the invention is a product of substantially or totally non-sequential reaction of the following components:
(i) at least one polyglycerol-3 comprising at least 25% by weight of diglycerol, at least 45% by weight of triglycerol and at least 10% by weight of tetraglycerol, in each case relative to the total weight of polyglycerol-3 in the form of a mixture;
(ii) at least one hydrogenated acid dimer containing at least 60% by weight of hydrogenated C36 diacid and from 5% to 25% by weight of hydrogenated C54 triacid, in each case relative to the total weight of hydrogenated acid; and
(iii) isostearic acid; in which the polymer has a combination of weight-average molecular mass >5000 Da and <15 000 Da, measured with GPC using linear polystyrene standards, and of viscosity of the pure polymer >100 000 mPa·s and <2 000 000 mPa·s at 25° C.; and in which the copolymer is also characterized by a total esterification of about 40%, an esterification with a hydrogenated acid dimer of about 27% and an esterification with a monoacid of about 13%.

In practice, given that the raw ingredients contain a range of polyglycerol units and a range of acid dimer and trimer contents, the above numbers can be adjusted using the actual (and not theoretical) hydroxyl fractions and carboxylic acid fractions, as are determined by methods such as mass spectrometry, NMR and liquid chromatography. The above esterification ranges are based on the ideal structure of the polyglycerol-3 and of the C36 acid dimer. The actual ranges may thus be slightly different from the values indicated above and may be calculated on the basis of these analytical values.

It is more practical to define the extent of the polymerization by the final acid number. The initial acid numbers, in the light of the distribution of the polyglycerol, monoacid and polyacid fractions present, can be reliably calculated using the actual acid number determined by the raw ingredient used.

For example, the initial total acid number (“AV”, which is commonly defined in mg of KOH/g of total reagent), is 135 AV. This comprises 68 AV for the acid dimer and 67 AV for the isostearic acid for a preferred embodiment containing 1 mol of polyglycerol-3, 0.5 mol of hydrogenated C36 acid dimer and 1 mol of isostearic acid. All the preferred ratio embodiments described above have a corresponding initial AV which can be calculated. When, during the polymerization reaction, the AV units are reduced, this ratio gives the percentage of conversion of the reaction from the total initial reactive acid fractions to the final residual acid fractions.

Thus, the degree of completion of the reaction is defined by (1—final AV)/initial AV.

In one embodiment, the polyesters of the invention have final acid numbers of from 0.1 to <25 mg of KOH/g of polymer.

In a preferred embodiment, the polyesters of the invention have final acid numbers of from 0.1 to <10 mg of KOH/g of polymer.

In a most preferred embodiment, the polyesters of the invention have final acid numbers of from 0.1 to <5 mg of KOH/g of polymer.

As the degree of completion of the reaction is defined by the equation 1—final AV/initial AV, the degree of completion of the reaction of such mixtures to give final polymer is >80%.

In a preferred embodiment, the degree of completion of the reaction of such mixtures to give final polymer is >90%.

In a most preferred embodiment, the degree of completion of the reaction of such mixtures to give final polymer is >95%.

In a preferred embodiment, the polyester of the invention is a product of reaction of a polyglycerol-3, of a hydrogenated C36 acid dimer and of isostearic acid in a mole ratio of 1/0.5/1, as described in Example 10 (copolymer) of US 2021/0259945.

According to a particularly preferred form of the invention, the composition comprises an oily solution comprising
a) a polyester obtained by reaction:
(i) of a polyglycerol-3; and
(ii) of a hydrogenated C36 acid dimer; and
(iii) of isostearic acid, components (i), (ii) and (iii) reacted being in a mole ratio of 1 mol of polyglycerol-3, 0.5 to 1 mol of acid dimer and from 0.1 to less than 2.0 mol of fatty acids; and
b) a caprylic/capric acid triglyceride;
said mixture having the INCI name: Diisostearoyl Polyglyceryl-3 Dimer Dilinoleate (and) Caprylic/Capric Triglyceride.

Such an oily solution is sold under the name SolAmaze Natural® by the company Nouryon, comprising 60% by weight, as active material, of polyester and 40% by weight of a caprylic/capric acid triglyceride, relative to the total weight of the oily solution. It is described in Example 10 (copolymer) and Example 28 (oil mixture) of the document US2021/0259945.

### Alkylcellulose

The composition according to the invention also comprises at least one alkylcellulose.

Ethylcellulose is a cellulose alkyl ether comprising a chain formed from β-anhydroglucose units linked together via acetal bonds. Each anhydroglucose unit contains three replaceable hydroxyl groups, all or some of these hydroxyl groups being able to react according to the following reaction:
RONa+R′Cl→ROR′+NaCl, where R represents a cellulose radical and R′ represents an ethyl radical.

Total substitution of the three hydroxyl groups would lead, for each anhydroglucose unit, to a degree of substitution of 3, in other words to a content of alkoxy groups of 54.88%.

The ethylcellulose polymers used in a cosmetic composition according to the invention are preferentially polymers with a degree of substitution with ethoxy groups ranging from 2.5 to 2.6 per anhydroglucose unit, in other words comprising a content of ethoxy groups ranging from 44% to 50%.

The ethylcellulose used in the composition according to the invention is more particularly in pulverulent form.

It is sold, for example, under the trade names Ethocel Standard from Dow Chemicals, notably including Ethocel Standard 7 FP Premium and Ethocel Standard 100 FP Premium. Other commercially available products, such as those sold by Ashland, Inc. under the trade names Aqualon EC type-K, type-N and type-T, preferably type-N, such as N7, N100, are particularly suitable for performing the invention.

Advantageously, the content of alkylcellulose, preferably of ethylcellulose, ranges from 1% to 30% by weight and preferably from 3% to 15% by weight relative to the total weight of the composition.

### Non-Volatile Oils

The composition according to the invention may optionally comprise at least one hydrocarbon-based or silicone non-volatile oil.

The term “hydrocarbon-based oil” refers to an oil mainly containing carbon and hydrogen atoms and possibly one or more functions chosen from hydroxyl, ester, ether and carboxylic functions. These oils are thus different from silicone oils.

For the purposes of the invention, the term “silicone oil” means an oil comprising at least one silicon atom, and notably at least one Si—O group.

According to a preferential embodiment, the composition in accordance with the invention comprises at least one non-volatile oil, the weight ratio of the total amount of non-volatile oil(s) to the amount of polyester preferably being less than 7.

According to a first variant, the composition according to the invention comprises at least one non-volatile hydrocarbon-based oil which is liquid at 20° C. and atmospheric pressure (1.013×10⁵ Pa).

The term “non-volatile oil” means an oil whose vapour pressure at 20° C. and atmospheric pressure is non-zero and is less than 2.66 Pa and more particularly less than 0.13 Pa. By way of example, the vapour pressure may be measured according to the static method or via the effusion method by isothermal thermogravimetry, depending on the vapour pressure of the oil (standard OCDE 104).

### Polar and Apolar Hydrocarbon-Based Oils

Among the non-volatile hydrocarbon-based oils, polar hydrocarbon-based oils, different from the polyester B), or apolar hydrocarbon-based oils may be suitable for use.

### Polar Hydrocarbon-Based Oils

The term “polar hydrocarbon-based oil” means that said oils comprise, in addition to carbon and hydrogen atoms, at least one oxygen atom. Thus, said hydrocarbon-based oil comprises at least one hydroxyl, ester, ether and/or carboxylic function.

The composition according to the invention may thus comprise at least one non-volatile polar hydrocarbon-based oil, more particularly chosen from:
saturated or unsaturated, linear or branched, C₁₀-C₂₆ fatty alcohols, preferably monoalcohols, which are preferably branched when they comprise at least 16 carbon atoms. More particularly, the fatty alcohol comprises from 10 to 24 carbon atoms and more preferentially from 12 to 22 carbon atoms;
ethers of formula ROR′ or carbonates of formula RO(CO)OR′, in which formulae, the groups R and R′, which may be identical or different, represent a saturated or unsaturated, branched or unbranched, hydrocarbon-based group comprising not more than 16 carbon atoms, preferably a C₃-C₁₆ group;
hydroxylated or non-hydroxylated plant oils;
optionally hydroxylated ester oils comprising from 1 to 4 ester functions, of which at least one of them, which is linear or branched, saturated, unsaturated or aromatic, comprises at least 8 carbon atoms;
liquid polyesters derived from the reaction of a monounsaturated or polyunsaturated acid dimer; the fatty acid comprising from 16 to 22 carbon atoms;
and mixtures thereof.

Preferably, the second oil is chosen from:
lauryl alcohol, isostearyl alcohol, oleyl alcohol, 2-butyloctanol, 2-undecylpentadecanol, 2-hexyldecyl alcohol, isocetyl alcohol and octyldodecanol, and mixtures thereof; preferably octyldodecanol;
dicaprylyl ether;
dipropyl carbonate, diethylhexyl carbonate, dicaprylyl carbonate, and C₁₄-C₁₅ dialkyl carbonate;
castor oil, olive oil, jojoba oil, ximenia oil, pracaxi oil, wheat germ oil, corn oil, sunflower oil, shea oil, sweet almond oil, macadamia oil, apricot kernel oil, soybean oil, rapeseed oil, groundnut oil, cottonseed oil, alfalfa oil, poppy oil, pumpkin oil, sesame oil, marrow oil, avocado oil, hazelnut oil, grape seed oil, blackcurrant oil, argan oil, evening primrose oil, millet oil, barley oil, linseed oil, quinoa oil, rye oil, safflower oil, candlenut oil, passionflower oil, musk rose oil, the liquid fraction of shea butter and the liquid fraction of cocoa butter, and mixtures thereof;
2-ethylhexyl palmitate, 2-octyldecyl palmitate, octyldodecyl neopentanoate, 2-octyldodecyl stearate, butyl stearate, 2-octyldodecyl erucate, C₁₂ to C₁₅ alkyl benzoates, 2-octyldodecyl benzoate, isocetyl isostearate, isostearyl isostearate, isononyl isononanoate, isopropyl palmitate, hexyl laurate, 2-hexyldecyl laurate, isopropyl myristate, 2-octyldodecyl myristate, diisostearyl malate, neopentyl glycol dicaprate, glyceryl tris(2-decyltetradecanoate), capric/caprylic acid triglycerides, C₁₈₋₃₆ acid triglycerides, glyceryl triheptanoate, glyceryl trioctanoate, glyceryl tris(2-decyltetradecanoate), triisostearyl citrate, tridecyl stearate, tridecyl trimellitate, pentaerythrityl tetrapelargonate, pentaerythrityl tetraisostearate, pentaerythrityl tetraisononanoate, pentaerythrityl tetrakis(2-decyltetradecanoate); isostearyl lactate, octyl hydroxystearate, octyldodecyl hydroxystearate;
polyesters having the following INCI names: dilinoleic acid/butanediol copolymer, dilinoleic acid/propanediol copolymer, dimer dilinoleyl dilinoleate;
and also mixtures thereof.

### Apolar Non-Volatile Hydrocarbon-Based Oils

Preferably, the apolar, non-volatile hydrocarbon-based oil may be chosen from linear or branched hydrocarbons of mineral, plant or synthetic origin, for instance:
liquid paraffin,
squalane, in particular of plant origin,
isoeicosane,
mixtures of saturated linear hydrocarbons, more particularly of C₁₅-C₂₈, such as the mixtures of which the INCI names are, for example, the following: C15-19 Alkane, C18-C21 Alkane, C21-C28 Alkane, for instance the products Gemseal 40, Gemseal 60 and Gemseal 120 sold by Total, and Emogreen L15 and L19 sold by SEPPIC,
hydrogenated or non-hydrogenated polybutenes, for instance products of the Indopol range sold by the company Ineos Oligomers,
hydrogenated or non-hydrogenated polyisobutenes, for instance the non-volatile compounds of the Parleam® range sold by the company Nippon Oil & Fats,
hydrogenated or non-hydrogenated polydecenes, for instance non-volatile compounds of the Silkflo range sold by the company Ineos, and Dekanex by the company IMCD,
and mixtures thereof.

### Non-Volatile Silicone Oils

The composition according to the invention may comprise at least one phenyl silicone non-volatile oil, optionally comprising at least one dimethicone fragment, or comprising at least one non-phenyl silicone non-volatile oil.

The term “phenyl(ated)” specifies that said oil includes, in its structure, at least one phenyl radical.

The term “dimethicone fragment” denotes a divalent siloxane group, the silicon atom of which bears two methyl radicals, this group not being located at one or both ends of the molecule. It may be represented by the following formula:

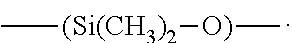

Preferably, the silicones do not contain a C₂-C₃ alkylene oxide group or a glycerolated group.

As non-volatile phenylated oil comprising at least one dimethicone fragment, mention may be made of the oils having the following INCI names: Trimethylsiloxyphenyl Dimethicone, Diphenyl Dimethicone, Tetramethyl Tetraphenyl Trisiloxane and also mixtures thereof, preferably Trimethylsiloxyphenyl Dimethicone. The Diphenyl Dimethicones are notably sold by the company Shin-Etsu under the names KF-54, KF54HV, KF-50-3000S, KF-53 d and KF-50-100CS. The Trimethylsiloxy Phenyl Dimethicones are sold, for example, by the company Wacker Chemie under the names Belsil PDM 1000 and Belsil PDM 20.

Among the non-volatile phenyl silicone oils not containing a dimethicone fragment, mention may be made of the compounds having the following INCI names: Phenyltrimethicone, Trimethyl Pentaphenyl Trisiloxanes, alone or as mixtures. As non-volatile, non-phenylated silicone oils that are suitable for performing the invention, mention may be made of those sold by the company Wacker under the Belsil DM range, by the company Dow Corning with the Xiameter PMX 200 Silicone Fluid range, and by the company Shin-Etsu with the KF-96 A range.

Representative examples of the non-volatile non-phenyl silicone oils include polydimethylsiloxanes and alkyl dimethicones. It should be noted that the term “dimethicone” (INCI name) corresponds to a polydimethylsiloxane (chemical name). Preferably, these non-volatile, non-phenyl silicone oils are chosen from polydimethylsiloxanes and alkyl dimethicones comprising at least one C₂-C₂₄ alkyl group, and also mixtures thereof. Thus, these oils may be chosen from dimethicone, cetyl dimethicone and stearyl dimethicone, alone or as mixtures. As non-volatile, non-phenyl silicone oils that are suitable for use, mention may be made of those sold by the company Wacker under the Belsil DM range, by the company Dow Corning with the Xiameter PMX 200 Silicone Fluid range, and by the company Shin-Etsu with the KF-96 A range. The alkyldimethicones may be sold, for example, under the trade names Abil Wax 9800 and Abil Wax 9801 from Evonik Goldschmidt, or Dowsil 2502 Cosmetic Fluid, Dowsil 2503 Cosmetic Wax, from Dow Corning; and mixtures thereof.

Preferably, the non-volatile oil is chosen from polar hydrocarbon-based oils other than the polyester B), in particular from ester oils. According to an even more preferred embodiment, the composition comprises at least one non-volatile polar hydrocarbon-based oil chosen from fatty alcohols, hydroxylated or non-hydroxylated plant oils, ester oils, which are optionally hydroxylated, comprising 1 to 4 ester functions, at least one of which is linear or branched, saturated, unsaturated or aromatic, comprising at least 8 carbon atoms. According to a preferred form, the non-volatile oil is chosen from triglycerides of fatty acids containing from 8 to 24 carbon atoms, and more particularly a caprylic/capric acid triglyceride (INCI name: Caprylic/capric triglyceride).

If the composition comprises any, the content of non-volatile, preferably hydrocarbon-based oil(s) ranges from 0.5% to 30% by weight, particularly from 2% to 8% by weight relative to the total weight of the composition.

### Dyestuffs

The composition according to the invention comprises at least one dyestuff.

According to a particular form of the invention, the dyestuff may be chosen from pulverulent dyestuffs, liposoluble dyes, water-soluble dyes and mixtures thereof.

### Pulverulent Dyestuffs

The pulverulent dyestuffs may be chosen from mineral pigments, organic pigments, nacres and mixtures thereof.

The term “pigments” means white or coloured, mineral or organic particles, which are insoluble in an aqueous medium, and which are intended to colour and/or opacify the resulting composition and/or deposit. These pigments may be white or coloured, and mineral and/or organic.

According to a particular embodiment, the pigments used according to the invention are chosen from mineral pigments.

The term “mineral pigment” refers to any pigment that satisfies the definition in Ullmann's encyclopaedia in the chapter on inorganic pigments. Among the mineral pigments that are useful in the present invention, mention may be made of zirconium oxide or cerium oxide, and also zinc oxide, iron oxide (black, yellow or red) or chromium oxide, manganese violet, ultramarine blue, chromium hydrate and ferric blue, titanium dioxide, and metal powders, for instance aluminium powder and copper powder. The following mineral pigments may also be used: Ta₂O₅, Ti₃O₅, Ti₂O₃, TiO, ZrO₂ as a mixture with TiO₂, ZrO₂, Nb₂O₅, CeO₂, ZnS.

The size of the pigment that is useful in the context of the present invention is generally greater than 100 nm and may range up to 10 μm, preferably from 200 nm to 5 μm and more preferentially from 300 nm to 1 μm.

According to a particular form of the invention, the pigments have a size characterized by a D[50] of greater than 100 nm and possibly ranging up to 10 μm, preferably from 200 nm to 5 μm and more preferentially from 300 nm to 1 μm.

The sizes are measured by static light scattering using a commercial MasterSizer 3000® particle size analyser from Malvern, which makes it possible to determine the particle size distribution of all of the particles over a wide range which may extend from 0.01 μm to 1000 μm. The data are processed on the basis of the standard Mie scattering theory. This theory is the most suitable for size distributions ranging from submicronic to multimicronic; it makes it possible to determine an “effective” particle diameter. This theory is notably described in the publication by Van de Hulst, H. C., Light Scattering by Small Particles, Chapters 9 and 10, Wiley, New York, 1957.

D[50] represents the maximum size exhibited by 50% by volume of the particles.

According to a particular form of the invention, the mineral pigment comprises a lipophilic or hydrophobic coating, said coating preferably being present in the oily phase of the composition according to the invention.

According to a particular embodiment of the invention, the pigments may be coated according to the invention with at least one compound chosen from metal soaps; N-acylamino acids or salts thereof; lecithin and derivatives thereof; isopropyl triisostearyl titanate; isostearyl sebacate; natural plant or animal waxes; polar synthetic waxes; fatty esters; phospholipids; and mixtures thereof.

According to a preferential embodiment, the pigments may be coated according to the invention with an N-acylamino acid or a salt thereof, which may comprise an acyl group containing from 8 to 22 carbon atoms, for instance a 2-ethylhexanoyl, caproyl, lauroyl, myristoyl, palmitoyl, stearoyl or cocoyl group.

The amino acid may be, for example, lysine, glutamic acid or alanine. The salts of these compounds may be the aluminium, magnesium, calcium, zirconium, zinc, sodium or potassium salts. Thus, according to a particularly preferred embodiment, the pigments may be coated with an N-acylamino acid derivative which may notably be a glutamic acid derivative and/or a salt thereof, and more particularly a stearoyl glutamate, for instance aluminium stearoyl glutamate. As examples of pigments treated with aluminium stearoyl glutamate, mention may be made of titanium dioxide pigments and black, red and yellow iron oxide pigments sold under the trade name NAI® by the company Miyoshi Kasei.

According to a preferential embodiment, the pigments may be coated according to the invention with isopropyl triisostearyl titanate. As examples of isopropyl titanium triisostearate (ITT)-treated pigments, mention may be made of titanium dioxide pigments and the black, red and yellow iron oxides sold under the trade names BWBO-12® (iron oxide C177499 and isopropyl titanium triisostearate), BWYO-12® (iron oxide C177492 and isopropyl titanium triisostearate) and BWRO-12® (iron oxide C177491 and isopropyl titanium triisostearate) by the company Kobo.

The pigments that may be used according to the invention may also be organic pigments.

The term “organic pigment” refers to any pigment that satisfies the definition in Ullmann's Encyclopedia in the chapter on organic pigments. The organic pigment may notably be chosen from nitroso, nitro, azo, xanthene, quinoline, anthraquinone, phthalocyanine, metal-complex type, isoindolinone, isoindoline, quinacridone, perinone, perylene, diketopyrrolopyrrole, thioindigo, dioxazine, triphenylmethane or quinophthalone compounds.

The organic pigment(s) may be chosen, for example, from carmine, carbon black, aniline black, melanin, azo yellow, quinacridone, phthalocyanine blue, sorghum red, the blue pigments codified in the Color Index under the references CI 42090, 69800, 69825, 73000, 74100 and 74160, the yellow pigments codified in the Color Index under the references CI 11680, 11710, 15985, 19140, 20040, 21100, 21108, 47000 and 47005, the green pigments codified in the Color Index under the references CI 61565, 61570 and 74260, the orange pigments codified in the Color Index under the references CI 11725, 15510, 45370 and 71105, the red pigments codified in the Color Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915 and 75470, and the pigments obtained by oxidative polymerization of indole or phenol derivatives as described in patent FR 2 679 771.

These pigments may also be in the form of composite pigments as described in patent EP 1 184 426. These composite pigments may notably be composed of particles including a mineral core at least partially covered with an organic pigment and at least one binder for fixing the organic pigments to the core.

The pigment may also be a lake. The term “lake” means insolubilized dyes adsorbed onto insoluble particles, the assembly thus obtained remaining insoluble during use.

The inorganic substrates onto which the dyes are adsorbed are, for example, alumina, silica, calcium sodium borosilicate or calcium aluminium borosilicate and aluminium.

Among the organic dyes, mention may be made of cochineal carmine. Mention may also be made of the products known under the following names: D&C Red 21 (CI 45 380), D&C Orange 5 (CI 45 370), D&C Red 27 (CI 45 410), D&C Orange 10 (CI 45 425), D&C Red 3 (CI 45 430), D&C Red 4 (CI 15 510), D&C Red 33 (CI 17 200), D&C Yellow 5 (CI 19 140), D&C Yellow 6 (CI 15 985), D&C Green 5 (CI 61 570), D&C Yellow 10 (CI 77 002), D&C Green 3 (CI 42 053), D&C Blue 1 (CI 42 090).

An example of a lake that may be mentioned is the product known under the name D&C Red 7 (CI 15 850:1).

Preferably, the composition according to the invention comprises at least one pulverulent dyestuff of mineral pigment type, in particular chosen from metal oxides, and more particularly chosen from coated or uncoated titanium dioxides or iron oxides and mixtures thereof.

The nacres may be chosen from white nacreous pigments such as mica coated with titanium or with bismuth oxychloride, coloured nacreous pigments such as titanium mica with iron oxides, titanium mica notably with ferric blue or chromium oxide, titanium mica with an organic pigment of the abovementioned type, and also nacreous pigments based on bismuth oxychloride.

Preferably, the pulverulent dyestuff(s) are present in the composition in a content ranging from 3% to 25% by weight, preferably from 5% to 20% by weight, more particularly from 8% to 15% by weight, relative to the total weight of the composition.

### Liposoluble or Water-Soluble Dyestuffs

A composition according to the invention may comprise at least one water-soluble or liposoluble dyestuff, preferably in a proportion of at least 0.01% by weight relative to the total weight of the composition.

For obvious reasons, this amount is liable to vary significantly with regard to the intensity of the desired colour effect and of the colour intensity afforded by the dyestuffs under consideration, and its adjustment clearly falls within the competence of a person skilled in the art.

The additional dyestuffs that are suitable for use in the invention may be liposoluble.

For the purposes of the invention, the term “liposoluble dyestuff” means any natural or synthetic, generally organic compound, which is soluble in an oily phase or in solvents that are miscible with a fatty substance, and which is capable of imparting colour.

As liposoluble dyes that are suitable for use in the invention, mention may notably be made of synthetic or natural liposoluble dyes, for instance DC Red 17, DC Red 21, DC Red 27, DC Green 6, DC Yellow 11, DC Violet 2, DC Orange 5, Sudan red, carotenes (β-carotene, lycopene), xanthophylls (capsanthin, capsorubin, lutein), palm oil, Sudan brown, quinoline yellow, annatto and curcumin.

The additional dyestuffs that are suitable for use in the invention may be water-soluble.

For the purposes of the invention, the term “water-soluble dyestuff” means any natural or synthetic, generally organic compound, which is soluble in an aqueous phase or water-miscible solvents and which is capable of imparting colour.

As water-soluble dyes that are suitable for use in the invention, mention may be made notably of synthetic or natural water-soluble dyes, for instance FDC Red 4, DC Red 6, DC Red 22, DC Red 28, DC Red 30, DC Red 33, DC Orange 4, DC Yellow 5, DC Yellow 6, DC Yellow 8, FDC Green 3, DC Green 5, FDC Blue 1, betanine (beetroot), carmine, copper chlorophyllin, methylene blue, anthocyanins (enocianin, black carrot, hibiscus, elder), caramel and riboflavin.

The water-soluble or liposoluble dye(s), if the composition comprises any, are preferably present in contents of less than 4% by weight, or even less than 2% by weight, more preferentially ranging from 0.01% to 2% by weight and better still from 0.02% to 1.5% by weight, relative to the total weight of the composition.

### Cosmetic Additives

The compositions according to the invention may also include additives commonly used in skincare and/or makeup products, such as active agents such as vitamins, for example vitamins A, E, C and B3, adenosine, hyaluronic acid and salts thereof; UV-screening agents; fillers; waxes; pasty compounds; hydrophilic gelling agents; film-forming agents other than alkylcellulose (in particular ethylcellulose); lipophilic gelling agents; fragrances; preserving agents; and mixtures thereof.

It is a matter of routine practice for a person skilled in the art to adjust the nature and amount of the additives present in the compositions in accordance with the invention such that the desired cosmetic properties thereof are not thereby affected.

### Fillers

The compositions in accordance with the invention may thus comprise at least one filler, notably making it possible to give them additional properties in terms of improved matiness, coverage, staying power and/or stability.

The term “filler” should be understood as meaning colourless or white solid particles of any shape which are provided in an insoluble form and dispersed in the medium of the composition. These particles give body or rigidity to the composition and/or softness and uniformity to the makeup.

The fillers may be mineral or organic.

Preferably, they may be chosen from natural fillers or fillers of natural origin.

The term “natural compound” means a compound that is obtained directly from the earth or the soil, or from plants or animals, via, where appropriate, one or more physical processes, for instance milling, refining, distillation, purification or filtration.

The term “compound of natural origin” means a natural compound that has undergone one or more additional chemical or industrial treatments, giving rise to modifications that do not affect the essential qualities of this compound and/or a compound predominantly comprising natural constituents that may or may not have undergone transformations. As non-limiting examples of additional chemical or industrial treatments bringing about modifications which do not affect the essential qualities of a natural compound, mention may be made of those permitted by the controlling bodies, such as Ecocert (Reference system for biological and ecological cosmetic products, January 2003), or defined in handbooks recognized in the field, such as “Cosmetics and Toiletries Magazine”, 2005, volume 120, 9:10.

The fillers used in the compositions according to the present invention may be in lamellar, globular or spherical form, in the form of fibres or in any other intermediate form between these defined forms.

The fillers according to the invention may or may not be surface-coated, and in particular they may be surface-treated with amino acids or any other substance that promotes the dispersion and compatibility of the filler in the composition.

### Mineral Fillers

Examples of mineral fillers that may be mentioned include talcs, natural or synthetic micas such as synthetic fluorphlogopites, silica, hydrophobic silica aerogels, hollow silica microspheres, kaolin, calcium carbonate, magnesium carbonate, hydroxyapatite, boron nitride, bismuth oxychloride, glass or ceramic microcapsules, or composites of silica and of titanium dioxide, such as the TSG® series sold by Nippon Sheet Glass.

### Organic Fillers

As examples of organic fillers, mention may be made of micronized natural waxes; metal soaps derived from organic carboxylic acids containing from 8 to 22 carbon atoms, preferably from 12 to 18 carbon atoms, for example zinc, magnesium or lithium stearate, zinc laurate or magnesium myristate; lauroyl lysine; or cellulose powders, such as the product sold by Daito in the Cellulobeads® range.

Preferably, the filler(s) are present in the composition in a content ranging from 0.5% to 20% by weight, preferably from 1% to 15% by weight, more particularly from 3% to 10% by weight, relative to the total weight of the composition.

### Waxes

The composition according to the invention may comprise at least one wax.

For the purposes of the present invention, the term “wax” means a lipophilic compound, which is solid at room temperature, with a reversible solid/liquid change of state, which has a melting point of greater than or equal to 30° C. that may be up to 120° C.

For the purposes of the invention, the melting point corresponds to the temperature of the most endothermic peak observed on thermal analysis (DSC) as described in the standard ISO 11357-3; 1999. The melting point of the wax may be measured using a differential scanning calorimeter (DSC), for example the calorimeter sold under the name DSC Q2000 by the company TA Instruments with the TA Universal Analysis software.

### the Measuring Protocol is as Follows:

A sample of 5 mg of wax placed in a crucible is subjected to a first temperature rise ranging from −20° C. to 120° C., at a heating rate of 10° C./minute, it is then cooled from 120° C. to −20° C. at a cooling rate of 10° C./minute and is finally subjected to a second temperature rise ranging from −20° C. to 120° C. at a heating rate of 5° C./minute.

During the second temperature rise, the melting point of the solid fatty substance is measured, which corresponds to the temperature of the most endothermic peak observed of the melting curve, representing the variation in the difference in power absorbed as a function of the temperature.

The enthalpy of fusion of the wax (ΔHf), corresponding to the integral of the entire melting curve obtained, may also be measured. This enthalpy of fusion of the wax is the amount of energy required to cause the compound to change from the solid state to the liquid state. It is expressed in J/g.

The waxes may be silicone waxes and, preferably, hydrocarbon-based waxes. They are also of plant, mineral, animal and/or synthetic origin.

In particular, the waxes have a melting point preferably greater than or equal to 35° C. and better still greater than or equal to 40° C.

### Apolar Waxes:

For the purposes of the present invention, the term “apolar hydrocarbon-based wax” means a wax consisting solely of carbon and hydrogen atoms and free of heteroatoms, for instance N, O, Si, P, etc.

As examples of apolar waxes that are suitable for use in the invention, mention may notably be made of hydrocarbon-based waxes, for instance microcrystalline waxes, paraffin waxes, ozokerite, polymethylene waxes, polyethylene waxes and microwaxes, notably polyethylene waxes.

### Polar Waxes

The polar waxes may notably be hydrocarbon-based or silicone waxes.

For the purposes of the present invention, the term “polar hydrocarbon-based wax” means a wax of which the chemical structure is formed essentially of, or even consists of, carbon and hydrogen atoms, and which comprises at least one heteroatom more particularly chosen from oxygen, optionally nitrogen, or mixtures thereof. It may thus contain alcohol, ester, ether, carboxylic acid, amine and/or amide groups.

The term “silicone wax” means an oil comprising at least one silicon atom and notably comprising Si—O groups.

According to a first preferred embodiment, the polar wax is a hydrocarbon-based wax.

A wax chosen from ester waxes and alcohol waxes is preferred as polar hydrocarbon-based wax.

According to the invention, the term “ester wax” means a wax comprising at least one ester function. The ester waxes may also be hydroxylated.

According to the invention, the term “alcohol wax” means a wax comprising at least one alcohol function, i.e. comprising at least one free hydroxyl (OH) group.

Use may notably be made, as ester wax, alone or as mixtures, of:
i) waxes of formula R₁COOR₂ in which R₁ and R₂ represent linear, branched or cyclic aliphatic chains, the number of atoms of which ranges from 6 to 50, notably from 10 to 50, which may contain a heteroatom, for instance O or N, and the melting point of which ranges more particularly from 30° C. to 120° C. In particular, use may be made, as ester wax, of a C₂₀-C₄₀ alkyl (hydroxystearyloxy)stearate (the alkyl group comprising from 20 to 40 carbon atoms), alone or as a mixture, or a C₂₀-C₄₀ alkyl stearate. Such waxes are notably sold under the names Kester Wax K 82 P®, Hydroxypolyester K 82 P®, Kester Wax K 80 P® or Kester Wax K82H by the company Koster Keunen. Use may also be made of stearyl heptanoate and stearyl caprylate and mixtures thereof;
ii) bis(1,1,1-trimethylolpropane) tetrastearate,
iii) diester waxes of a dicarboxylic acid, of general formula
R³—(—OCO—R⁴—COO—R⁵), in which R³ and R⁵ are identical or different, preferably identical, and represent a C₄-C₃₀ alkyl group and R⁴ represents a linear or branched C₄-C₃₀ aliphatic group which may or may not contain one or more unsaturations. Preferably, the C₄-C₃₀ aliphatic group is linear and unsaturated;
iv) mention may also be made of the waxes obtained by catalytic hydrogenation of animal or plant oils notably containing linear or branched C₈-C₃₂ fatty chains, for instance hydrogenated jojoba oil, hydrogenated sunflower oil, hydrogenated castor oil or hydrogenated coconut kernel oil, and also the waxes obtained by hydrogenation of castor oil esterified with cetyl alcohol, such as those sold in the Phytowax Castor range, for example Phytowax Castor 22L73®, or else the waxes obtained by hydrogenation of olive oil esterified with stearyl alcohol, such as those of the Phytowax Olive range, for example Phytowax Olive 18L57, sold by the company Sophim. Such waxes are notably described in patent application FR2792190;
v) waxes corresponding to the partial or total esters, preferably total esters, of a saturated, optionally hydroxylated, C₁₆-C₃₀ carboxylic acid with glycerol. The term “total esters” means that all the hydroxyl functions of glycerol are esterified. Examples that may be mentioned include trihydroxystearine (or glyceryl trihydroxystearate), tristearine (or glyceryl tristearate) and tribehenine (or glyceryl tribehenate), alone or as a mixture. Among the suitable compounds, mention may be made of triesters of glycerol and of 12-hydroxystearic acid, or hydrogenated castor oil, for instance Thixcin R and Thixcin E sold by Elementis Specialties,
v) Mention may also be made of waxes of animal or plant origin, such as beeswax, synthetic beeswax, carnauba wax, candelilla wax, rice bran wax, ouricury wax, esparto grass wax, cork fibre wax, sugar cane wax, Japan wax, sumac wax, montan wax, orange wax, laurel wax, or sunflower wax, in particular refined sunflower wax;
vii) mention may also be made of natural or synthetic polyalkylated or polyglycerolated hydrocarbon-based waxes, of animal or plant origin; the number of (C₂-C₄) oxyalkylene units may range from 2 to 100, the number of glycerol units may range from 1 to 20. Examples that may be mentioned include polyoxyethylenated beeswaxes, such as PEG-6 beeswax or PEG-8 beeswax; polyoxyethylenated carnauba waxes, such as PEG-12 carnauba; polyoxyethylenated or polyoxypropylenated and hydrogenated or non-hydrogenated lanolin waxes, such as PEG-30 lanolin or PEG-75 lanolin; PPG-5 lanolin wax glyceride; polyglycerolated beeswaxes, notably polyglyceryl-3 beeswax, the Acacia Decurrens/Jojoba/Sunflower Seed Wax/Polyglyceryl-3 Esters mixture, polyglycerolated plant waxes, such as mimosa, jojoba or sunflower waxes, and mixtures thereof (Acacia Decurrens/Jojoba/Sunflower Seed Wax Polyglyceryl-3 Esters),

According to another embodiment, the polar wax may be an alcohol wax. As alcohol waxes, mention may be made of mixtures of saturated linear C₃₀-C₅₀ alcohols, for instance the wax Performacol 550 Alcohol from New Phase Technologies, stearyl alcohol and cetyl alcohol, or mixtures thereof.

### Silicone Waxes

As silicone wax, examples that may be mentioned include mixtures comprising a compound of C30-45 Alkyldimethylsilyl Polypropylsilsesquioxane (INCI name) type, for example the product Dow Corning SW-8005 C30 Resin Wax sold by the company Dow Corning. Mention may also made of mixtures comprising a compound of the C30-45 Alkyl Methicone (INCI name) type, for instance the product Dow Corning® AMS-C30 Cosmetic Wax. Mention may also be made of siliconized beeswax.

Preferably, if the composition comprises any, the wax is chosen from hydrocarbon-based waxes. More particularly, it is chosen from apolar waxes; polar hydrocarbon-based waxes such as waxes of animal or plant origin, waxes of animal or plant origin obtained by catalytic hydrogenation of animal or plant oils; alcohol waxes; and also mixtures thereof; and preferably from apolar hydrocarbon-based waxes, alone or as mixtures.

The wax content, if the composition comprises any, advantageously ranges from 1% to 20% by weight, in particular from 5% to 15% by weight, relative to the total weight of the composition.

### Pasty Compounds

The composition according to the invention may also comprise at least one compound which is pasty at room temperature and atmospheric pressure.

For the purposes of the present invention, the term “pasty” refers to a lipophilic compound with a reversible solid/liquid change of state, notably having in the solid state an anisotropic crystal organization, and comprising at room temperature a liquid fraction and a solid fraction.

In other words, the starting melting point of the pasty compound may be lower than room temperature. The liquid fraction of the pasty compound, measured at room temperature, may represent 9% to 97% by weight of the pasty compound. This fraction that is liquid at room temperature preferably represents between 15% and 85%, more preferably between 40% and 85%, by weight.

The melting point of the pasty fatty substance is determined according to the same principle as that described in detail previously for the waxes.

In the case of a pasty compound, the measurement protocol is, however, as follows:

A sample of 5 mg of pasty fatty substance placed in a crucible is subjected to a first temperature rise passing from −20° C. to 100° C., at a heating rate of 10° C./minute, it is then cooled from 100° C. to −20° C. at a cooling rate of 10° C./minute and it is finally subjected to a second temperature rise passing from −20° C. to 100° C. at a heating rate of 5° C./minute.

The melting point of the pasty fatty substance is the value of the temperature corresponding to the top of the peak on the curve representing the variation in the difference in power absorbed as a function of the temperature.

It should be noted that the liquid fraction by weight of the pasty fatty substance at room temperature is equal to the ratio of the heat of fusion consumed at room temperature to the heat of fusion of the pasty fatty substance.

The heat of fusion of the pasty fatty substance is the heat consumed by said substance in order to pass from the solid state to the liquid state. The pasty fatty substance is said to be in the solid state when all of its mass is in crystalline solid form. The pasty fatty substance is said to be in the liquid state when all of its mass is in liquid form.

The heat of fusion of the pasty fatty substance is the amount of energy required to make the pasty fatty substance change from the solid state to the liquid state. It is expressed in J/g. The heat of fusion of the pasty fatty substance is equal to the area under the curve of the thermogram obtained.

The pasty compound may in particular be chosen from synthetic pasty compounds and fatty substances of plant origin.

The pasty compound(s) may in particular be chosen from:
lanolin and derivatives thereof, such as lanolin alcohol, oxyethylenated lanolins, acetylated lanolin, lanolin esters such as isopropyl lanolate, and oxypropylenated lanolins,
petroleum jelly (also known as petrolatum),
ethers of pentaerythritol and of C₂-C₄, polyalkylene glycol, for example the compounds having the INCI names below: PEG-5 Pentaerythrityl Ether, PPG-5 Pentaerythrityl Ether, and mixtures thereof. Mention may be made, for example, of the mixture sold under the name Lanolide by the company Vevy,
liposoluble polyethers resulting from polyetherification between one or more C₂-C₁₀₀ and preferably C₂-C₅₀ diols. Among the liposoluble polyethers, consideration is given in particular, to copolymers of ethylene oxide and/or of propylene oxide with long-chain C₆-C₃₀ alkylene oxides, more preferably such that the weight ratio of the ethylene oxide and/or propylene oxide to alkylene oxides in the copolymer is from 5:95 to 70:30. In this family, mention will notably be made of the product having the INCI name PEG-45/Dodecyl Glycol Copolymer sold, for example, under the brand name Elfacos ST9 by the company Akzo Nobel,
esters resulting from the condensation of a preferably saturated, linear or branched, C₆-C₁₀ dicarboxylic acid and of an ester of diglycerol and of optionally hydroxylated, preferably saturated, linear or branched, C₆-C₂₀ monocarboxylic acids, in particular the diester obtained by condensation of adipic acid and of a mixture of esters of diglycerol with a mixture of C₆-C₂₀ fatty acids, such as caprylic acid, capric acid, stearic acid, isostearic acid and 12-hydroxystearic acid, notably sold under the reference Softisan® 649 by the company Cremer Oleo (INCI name: Bis-Diglyceryl Polyacyladipate-2),
triglycerides of fatty acids which are optionally hydrogenated (totally or partially), saturated or unsaturated, linear or branched, optionally mono- or polyhydroxylated, preferably C₁₂-C₁₈; for instance the glycerides of saturated C₁₂-C₁₈ fatty acids sold under the name Softisan 100® by the company Cremer Oleo (INCI name: Hydrogenated Coco-Glycerides),
esters of diol dimer, or of polyol, and of diacid dimer, for instance:
esters of dimer of dilinoleyl alcohol and of dilinoleic acid, the hydroxyl groups of which are esterified with a mixture of phytosterols, of behenyl alcohol and of isostearyl alcohol, for example the ester sold under the name Plandool G by the company Nippon Fine Chemical (INCI name: Bis-Behenyl/Isostearyl/Phytosteryl Dimer Dilinoleyl Dimer Dilinoleate);
esters of dilinoleic acid and of a mixture of phytosterols, of isostearyl alcohol, of cetyl alcohol, of stearyl alcohol and of behenyl alcohol, for example the ester sold under the name Plandool H or Plandool S by the company Nippon Fine Chemical (INCI name: Phytosteryl/Isostearyl/Cetyl/Stearyl/Behenyl Dimer Dilinoleate);
butters of plant origin, such as mango butter, such as the product sold under the name Lipex 203 by the company Aarhuskarlshamn, shea butter, in particular the product whose INCI name is Butyrospermum Parkii Butter, such as the product sold under the reference Sheasoft® by the company Aarhuskarlshamn, cupuacu butter (Rain Forest RF3410 from the company Beraca Sabara), murumuru butter (Rain Forest RF3710 from the company Beraca Sabara), cocoa butter; and also orange wax, for instance the product sold under the reference Orange Peel Wax by the company Koster Keunen,
totally or partially hydrogenated plant oils, for instance hydrogenated soybean oil, hydrogenated coconut kernel oil, hydrogenated rapeseed oil, mixtures of hydrogenated plant oils such as the mixture of hydrogenated soybean, coconut kernel, palm and rapeseed plant oil, for example the mixture sold under the reference Akogel® by the company Aarhuskarlshamn (INCI name Hydrogenated Vegetable Oil), the trans-isomerized partially hydrogenated jojoba oil manufactured or sold by the company Desert Whale under the commercial reference Iso-Jojoba-50®, partially hydrogenated olive oil, for instance the compound sold under the reference Beurrolive by the company Soliance,
hydrogenated castor oil esters, such as hydrogenated castor oil dimer dilinoleate, for example Risocast-DA-L sold by Kokyu Alcohol Kogyo, and hydrogenated castor oil isostearate, for example Salacos HCIS (V-L) sold by Nisshin Oil,
and mixtures thereof.

If the composition comprises at least one pasty compound, its/their content ranges from 0.5% to 20% by weight, preferably from 1% to 15% by weight, relative to the total weight of the composition.

### Lipophilic Gelling Agents

The composition according to the invention may optionally comprise at least one lipophilic gelling agent.

As lipophilic gelling agents, examples that may be mentioned include lipophilic clays.

The term “lipophilic clay” refers to any clay that is liposoluble or lipodispersible in the oily phase of the composition.

Clay denotes a material based on hydrated silicates and/or aluminosilicates, of lamellar structure.

The clays may be natural or synthetic, and they are made lipophilic by treatment with an alkylammonium salt such as a C10 to C22 ammonium chloride, in particular stearalkonium chloride or distearyldimethylammonium chloride.

They may be chosen from bentonites, in particular bentonites, hectorites and montmorillonites, beidellites, saponites, nontronites, sepiolites, biotites, attapulgites, vermiculites and zeolites.

They are preferably chosen from hectorites and bentonites.

For example, use may be made of a lipophilic clay chosen from hydrophobically modified bentonites and hydrophobically modified hectorites, notably modified with a C10 to C22 quaternary ammonium chloride, such as:
a bentonite modified with stearalkonium chloride, such as the commercial products sold under the name Claytone AF®, Garamite VT®, Tixogel® LG-M, Tixogel® MP 250 Tixogel® VZ and Tixogel® VZ-V XR, by the company BYK Additives Inc; or the commercial products sold under the name Viscogel® B3, Viscogel® B4, Viscogel® B7, Viscogel® B8, Viscogel® ED, Viscogel® GM, Viscogel® S4 and Viscogel® SD by the company Bentec S.P.A;
a bentonite modified with stearalkonium chloride in the presence of at least propylene carbonate and at least one oil, such as the commercial products Dub Velvet Gum® from the company Stéarinerie Dubois Fils, Miglyol Gel T® from the company Cremer Oleo, Tixogel® CGT 6030, Tixogel® DBA 6060, Tixogel® FTN, TIXOGEL® FTN 1564, Tixogel® IPM, Tixogel® LAN, Tixogel® LAN 1563 from the company BYK Additives Inc.;
a hectorite modified with distearyldimethylammonium chloride (INCI name: Disteardimonium Hectorite), for instance the product sold under the name Bentone® 38VCG Rheological Additive by the company Elementis Specialties;
a hectorite modified with distearyldimethylammonium chloride in the presence of at least propylene carbonate or triethyl citrate and of at least one oil, such as the commercial products sold under the name Bentone® Gel DOA V, Bentone® Gel EUG V, Bentone® Gel IHD V, Bentone® Gel ISD V, Bentone® Gel MIO V, Bentone® Gel PTM V, Bentone® SS-71 V, Bentone® VS-5 PC V or Bentone® VS-5 by the company Elementis Specialities; the commercial products sold under the name Creagel Bentone CPS/Hectone CPS or Creagel Bentone ID/Hectone ID by the company Créations Couleurs; the commercial products sold under the name NS Gel DM1®, NS Gel PTIS® or NS MGel 1152® by the company Next Step Laboratories Stop.

As lipophilic gelling agents, mention may also be made of esters of dextrin and of fatty acid, in particular C12 to C24, preferably C14 to C18, fatty acid, or mixtures thereof. More preferentially, the dextrin ester is an ester of dextrin and of a C12-C18 and in particular C14-C18 fatty acid.

Preferably, the lipophilic gelling agent may be present in the composition in concentrations preferably ranging from 0.1% to 5% by weight, and more preferentially from 0.5% to 3% by weight relative to the total weight of the composition.

Throughout the description, including the claims, the expression “comprising a” should be understood as being synonymous with “comprising at least one”, unless otherwise specified.

The expressions “between . . . and . . . ” and “ranging from . . . to . . . ” should be understood as meaning limits included, unless otherwise specified.

In addition, the sum of the amounts of the ingredients of the composition represents 100% by weight of the composition.

The invention is illustrated in greater detail by the examples presented below.

Unless otherwise indicated, the amounts indicated are expressed as mass percentages.

The examples that follow are presented as non-limiting illustrations of the invention.

### EXAMPLES

Composition 1 according to the invention, containing 10% by weight of a mixture of polyester Diisostearoyl Polyglyceryl-3 Dimer Dilinoleate (6% by weight) and of Caprylic/Capric Triglyceride (4% by weight), was prepared.

Comparative Examples 1a and 1b outside the invention were prepared:

Comparative Example 1a is of identical composition except that the polyester is replaced with the same content of Bis-Diglyceryl Polyacyladipate-2.

Comparative Example 1b is of identical composition except that the polyester is replaced with the same content of Hydrogenated Castor Oil Dimer Dilinoleate.

The compositions are detailed in the table below.

TABLE 1

| Ingredients (chemical name | Invention | Comparative | Comparative |
|---|---|---|---|
| or INCI name) | 1 | 1a | 1b |
| Isododecane | 34.15 | 34.15 | 34.15 |
| Absolute ethanol | 34.15 | 34.15 | 34.15 |
| Ethylcellulose | 11.7 | 11.7 | 11.7 |
| (Aqualon EC N7 ® - Ashland) | | | |
| Diisostearoyl Polyglyceryl-3 | 10 | | |
| Dimer Dilinoleate (and) | | | |
| Caprylic/Capric Triglyceride | | | |
| (SolAmaze Natural ®-Nouryon) | | | |
| Bis-Diglyceryl Polyacyladipate- | | 10 | |
| 2 (Softisan 649 ® - Sasol) | | | |
| Hydrogenated Castor Oil Dimer | | | 10 |
| Dilinoleate (Risocast-DA-L ® - | | | |
| Kokyu Alcohol Kogyo) | | | |
| Red 7/CI 15850 | 10 | 10 | 10 |

### Composition Preparation Method:

A portion of the isododecane, the alcohol and the polyester (SolAmaze, Softisan or Risocast) were mixed in a beaker. The mixture was stirred with a Rayneri flocculator at 500 rpm for 2 minutes.

The ethylcellulose was introduced as a fine mist while stirring with the deflocculator at 500 rpm (into the vortex) and the mixture was then left stirring for 10 minutes.

The pigment, ground beforehand using a three-roll mill, was added with the remaining isododecane.

### Evaluation of the Compositions:

### Protocol for Evaluating the Staying Power

Each composition was applied to an Erichsen contrast chart, using a spreader, as a deposit with a thickness of 24.5 μm, over a width of at least 6 cm, and was left to dry on a hotplate at 32° C. for 40 minutes.

Three thin strips of Wypall® fabric (Kimberley Clark) of 2 cm were placed on the deposit without them overlapping:
the first strip is dry,
the second strip is impregnated with distilled water (0.1 ml),
the third strip is impregnated with olive oil (0.1 ml).

The film spreader weighted with a weight of 2 kg was placed on all of the thin strips, and the assembly was moved over the film.

The state of the deposit was observed.

The operation was repeated for each of the compositions prepared.

### Grading of the Staying Power

TABLE 2

| Note | State of the deposit |
|---|---|
| 1 | Total or almost total removal of the deposit from the tested area; |
| | the surface of the support shows through in many places. |
| 2 | Partial removal of the deposit from the tested area; the surface of |
| | the support shows through in some places. |
| 3 | Slight removal of the deposit, leaving the support visible in a few |
| | places. |
| 4 | No substantial change in the colour of the deposit (homogeneity, |
| | colour) |
| 5 | No variation in the appearance of the deposit (homogeneity, |
| | colour) |

### Results

TABLE 3

| Invention | Comparative | Comparative |
|---|---|---|
| 1 | 1a | 1c |

Deposit on application

| | | |
|---|---|---|
| Homogeneous, | Homogeneous, a little | Homogeneous, |
| intense | less intense | intense |

Deposit after the staying power tests

| | | | |
|---|---|---|---|
| Applied dry | 5 | 5 | 3 |
| With water | 5 | 5 | 3 |
| With oil | 5 | 1 | 3 |

The above results confirm the superiority of the composition according to the invention. The composition notably made it possible to obtain a homogeneous and intense deposit, the dry resistance, resistance to water and resistance to oil of which were significantly improved.

1. Composition for making up and/or caring for the skin and/or the lips, comprising, notably in a physiologically acceptable medium:
A) at least one volatile solvent;
B) at least one polyester which is the product of reaction of components (i), (ii) and (iii) below:
(i) at least one polyglycerol-3;
(ii) at least one acid dimer; and
(iii) at least one fatty monoacid containing from 8 to 30 carbon atoms, components (i), (ii) and (iii) reacted being in a mole ratio of 1 mol of polyglycerol-3, 0.5 to 1 mol of acid dimer and from 0.1 to less than 2.0 mol of fatty monoacid; and
C) at least one alkylcellulose, the alkyl group of which is C2-C3, preferably ethylcellulose;
D) at least one dyestuff.

2. Composition according to claim 1, characterized in that the volatile solvent is chosen from monoalcohols including from 2 to 6 carbon atoms, volatile oils, preferably apolar hydrocarbon-based oils, and mixtures thereof.

3. Composition according to claim 1, characterized in that the composition comprises a mixture of volatile hydrocarbon-based oil(s) and of monoalcohol; in particular, the volatile hydrocarbon-based oil is chosen from isododecane, a mixture of undecane and tridecane, and mixtures thereof, and the monoalcohol is ethanol.

4. Composition according to claim 2, characterized in that the amount of monoalcohol(s) ranges from 2% to 60% by weight, preferably from 4% to 50% by weight and even more preferentially from 6% to 40% by weight.

5. Composition according to claim 2, characterized in that the amount of volatile oil(s) is less than 60% by weight and preferably less than 50% by weight relative to the total weight of said composition.

6. Composition according to claim 1, characterized in that the weight ratio of the amount of volatile oil(s) to the amount of monoalcohol(s) is less than 3.5 and even more preferentially less than 1.5.

7. Composition according to claim 1, characterized in that the polyester is a product of substantially or totally non-sequential reaction.

8. Composition according to claim 1, characterized in that the polyester is prepared by a one-step process which involves the introduction of all the reagents into a reaction vessel and subsequently the induction of an entirely random addition of the acid dimer and of the isostearic acid to the polyglycerol-3.

9. Composition according to claim 1, characterized in that the polyglycerol-3 is composed of at least 40% by weight, or of at least 45% by weight, or of at least 50% by weight, relative to the total weight of the polyglycerol-3, of a combination of diglycerol and of triglycerol.

10. Composition according to claim 1, characterized in that the polyglycerol-3 in the form of a mixture is composed of at least 20% by weight, or of at least 25% by weight, of diglycerol; at least 15% by weight, or at least 18% by weight, of triglycerol; at least 10% by weight, or at least 12% by weight, of tetraglycerol; in which all the weight percentages are based on a total content of the polyglycerol-3 in the form of a mixture.

11. Composition according to claim 1, characterized in that polyglycerol-3 under the form of a mixture comprises at least 25% by weight of diglycerol, at least 45% by weight of triglycerol and at least 10% by weight of tetraglycerol, relative to the total weight of the polyglycerol-3 in the form of a mixture.

12. Composition according to claim 1, characterized in that the polyester is a product of reaction of polyglycerol-3, of hydrogenated C36 acid dimer and of isostearic acid in a mole ratio of 1/0.5/1.

13. Composition according to claim 1, characterized in that the amount of polyester active material ranges from 1% to 50% by weight and more preferentially from 3% to 20% by weight relative to the total weight of the composition.

14. Composition according to claim 1, characterized in that the polyester is in an oily solution comprising
a) a polyester obtained by reaction:
(i) of a polyglycerol-3; and
(ii) of a hydrogenated C36 acid dimer; and
(iii) of isostearic acid, components (i), (ii) and (iii) reacted being in a mole ratio of 1 mol of polyglycerol-3, 0.5 to 1 mol of acid dimer and from 0.1 to less than 2.0 mol of fatty acids; and
b) a caprylic/capric acid triglyceride, said mixture more particularly having the INCI name: Diisostearoyl Polyglyceryl-3 Dimer Dilinoleate (and) Caprylic/Capric Triglyceride.

15. Composition according to claim 14, characterized in that the oily solution contains the polyester in a concentration of from 10% to 99% by weight, more preferentially from 30% to 90% by weight and more particularly from 50% to 80% by weight, relative to the total weight of the mixture.

16. Composition according to claim 14, characterized in that the oily solution comprises 40% by weight of caprylic/capric acid triglyceride and 60% by weight of polyester of polyglycerol-3, of hydrogenated C36 acid dimer and of isostearic acid in a mole ratio of 1/0.5/1.

17. Composition according to claim 1, characterized in that the content of alkylcellulose, preferably of ethylcellulose, ranges from 1% to 30% by weight and preferably from 3% to 15% by weight relative to the total weight of the composition.

18. Composition according to claim 1, characterized in that the composition optionally comprises at least one non-volatile hydrocarbon-based oil, which is preferably polar, other than the polyester B), in particular chosen from ester oils.

19. Composition according to claim 18, characterized in that the content of non-volatile, preferably hydrocarbon-based oil(s) ranges from 0.5% to 30% by weight and more particularly from 2% to 8% by weight, relative to the total weight of the composition.

20. Composition according to claim 18, characterized in that the weight ratio of the total amount of non-volatile oil(s) to the amount of polyester is less than 7.

21. Composition according to claim 1, characterized in that the composition is liquid.

22. Composition according to claim 1, characterized in that the composition comprises less than 5% by weight of water, more particularly less than 2% by weight of water and preferably less than 0.5% by weight of water relative to the total weight of the composition.

23. Composition according to claim 1, characterized in that the dyestuff is chosen from pulverulent dyestuffs, liposoluble dyes, water-soluble dyes and mixtures thereof.

24. Composition according to claim 23, characterized in that the pulverulent dyestuffs are chosen from mineral pigments, organic pigments, nacres, and mixtures thereof.

25. Composition according to claim 23, characterized in that the pulverulent dyestuff(s) are present in a content ranging from 3% to 25% by weight, preferably from 5% to 20% by weight and more particularly from 8% to 15% by weight relative to the total weight of the composition.

26. Composition according to claim 1, characterized in that the composition optionally comprises at least one additive chosen from waxes, pasty lipophilic compounds, active agents such as vitamins, for example vitamins A, E, C and B3, adenosine, hyaluronic acid and salts thereof; UV-screening agents; fillers; film-forming agents; lipophilic gelling agents; fragrances; preserving agents; and mixtures thereof.

27. Process for making up and/or caring for the lips, characterized in that it comprises at least the application to the lips of a composition as defined in claim 1.
